(19) United States
(12) Patent Application Publication
(10) Pub. No.: US 2015/0246083 A1
(43) Pub. Date: Sep. 3, 2015

(54) GENETICALLY MODIFIED PROBIOTIC FOR THE TREATMENT OF PHENYLKETONURIA (PKU) DISEASE

(71) Applicant: University of North Texas, Denton, TX (US)

(72) Inventors: Katherine Deming, Irving, TX (US); Michael Allen, Denton, TX (US); Ione Hunt von Herbing, Denton, TX (US)

(21) Appl. No.: 14/417,176

(22) PCT Filed: Jul. 26, 2013
(86) PCT No.: PCT/US2013/052200

§ 371 Date: Jan. 26, 2015

Related U.S. Application Data
(60) Provisional application No. 61/676,461, filed on Jul. 27, 2012.

(51) Int. Cl. A61K 35/747; A61K 38/51

(52) U.S. Cl. CPC A61K 35/747; A61K 38/51; A61K 2035/115

(57) ABSTRACT

A GMP adapted to provide the PAL gene for the treatment of PKU when administered orally. The GMP of the present invention may include a probiotic, a PAL gene to be expressed using the probiotic, wherein the PAL gene is functionally attached to a promoter and a ribosome binding site, and may be codon-optimized for expression in a certain host organism. A method of treating the metabolic disease of PKU by oral administration and ingestion of a GMP is also provided.

### BACKGROUND

The present invention relates to a genetically modified probiotic (GMP), and more specifically to a GMP adapted to provide the phenylalanine ammonia-lyase (PAL) gene or other phenylalanine-degrading enzyme for the treatment of phenylketonuria (PKU). The GMP is herein referred to as PHEnominal.

### ADVANTAGES OVER CONVENTIONAL TECHNOLOGIES

PKU is a disease that renders the patient incapable of digesting the amino acid phenylalanine, resulting in a subsequent accumulation in the blood causing toxicity.

Several technologies have been developed for the treatment of PKU. First, liver and/or hepatocyte transplants have been used. A regular transplant is not considered an acceptable treatment by insurance companies as the cost of treatment for PKU transfers to the cost of treatment to prevent organ rejection.

Second, gene therapy techniques have been utilized for the treatment of PKU. Gene therapy methods have yet to break the barrier of gene silencing. In gene silencing, a eukaryotic cell detects genes of a viral source, such as a gene therapy vector, and methylates the DNA to silence it. Additionally, after the “Bubble boy” syndrome incident (children developing leukemia following gene therapy for a terminal illness), gene therapy has not gained wide spread acceptance or regulatory approval in the USA.

Finally, several other treatments have been tried, without the level of cost-effectiveness required for wide-spread adoption.

Other treatments for PKU patients are made difficult by the many co-factors and co-enzymes required for PAH (the deficient enzyme) to function. Current dietary therapies cost approximately $40,000.00 per year for a teen or adult. Co-factor therapy, which supplies an excess of PAH's co-factor, reduces phenylalanine (phe) in some of the patients. However many patients (40-50%) see no improvements, or improvement is not sufficient to warrant the cost of co-factor therapy. The cofactor therapy by the name of Kuvan®, is also very expensive ($40,000.00 per year for an adult or teen). Responses from this treatment range from no change to complete control of phenylalanine levels. It is thought that the patient's level of response to this therapy is dictated by the exact genetic mutation they carry.

PEG-PAL, is currently under clinical trials. For this treatment, PAL is produced by bacteria, purified, and coated in polyethylene glycol (PEG). The resulting PEG-PAL is injected subcutaneously into the patient, and enters the blood stream. Once in the blood, the PEG coating helps protect PAL from immunodetection while allowing the enzyme to degrade serum phenylalanine. Although cost estimates have not been released, similar therapies (PEG-Adenosine deaminase) are quite expensive. Additionally, complications of this therapy have been detected in phase 2 clinical trials.

Yeast PAL in microcapsules has also been studied as a potential treatment. This study used rats injected with phenylalanine rather than a true disease model. Yeast PAL has lower activity (cannot compare exactly, due to notation used), and of further concern, yeast PAL is nearly as good at catabolizing tyrosine as phe (0.67 micromoles/min per unit enzyme for tyrosine and 1.4 micromoles/min per unit enzyme at catabolizing phe). The latter is of particular concern since PKU patients also are unable to synthesize the essential amino acid tyrosine from phe. Additionally, microcapsules were deemed too expensive to use as a PKU therapy.

Oral naked PAL enzyme has been developed, but was only used in mice due to the restrictions of treatment. Namely, the PAL enzyme could not survive the stomach to arrive in the intestine unless it was given by gastric gavage along with enough sodium bicarbonate to neutralize the stomach acid. Furthermore, once in the intestine, PAL is cleaved rapidly by proteases such as trypsin and chymotrypsin.

The only current option known to work as a treatment in all patients with PKU is dietary restriction of the ingestion of foods containing phe. No natural protein is free of phe, and only a handful of proteins have been identified as having low phe contents. To this end, restricting phe ingestion can be fostered by the ingestion of synthetic foods. These foods are reported as having repugnant odor and flavor, resulting in poor dietary compliance. In addition to a diet more limited than that of a person with gluten intolerance, and the cost of the synthetic food diet is similar to that of the co-factor therapy ($1,000.00 per month for infant formula, with costs increasing as the patient becomes an adult and requires more food). Insurance companies commonly deny assistance with the food expenses of this treatment.

The invention described herein meets the need for a cost-effective therapy for PKU, with tolerable side-effects and high efficacy.

### SUMMARY

The present invention relates to a GMP adapted to provide the PAL gene or other phenylalanine-degrading enzyme for the treatment of PKU. In certain embodiments, the GMP of the present invention comprises a probiotic, a PAL gene to be expressed using the probiotic, wherein the PAL gene is functionally attached to a promoter and a ribosome binding site, and may be codon-optimized for expression in a certain host organism.

The present invention further comprises a method of treating the metabolic disease of PKU by oral administration and ingestion of a GMP. The GMP described herein will produce the enzyme PAL in the intestine when administered orally. The genetically engineered PAL will digest ingested phenylalanine contained in most proteinous foods. The end result will be that phenylalanine obtained from ingestion is not absorbed into the blood stream and cannot build up to toxic levels.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the creation of the final functional mouse therapy shuttle vector starting with A) the E. coli plasmid pSLER1, B) addition of the pGT232 fragment to create shuttle vector ability, and C) final addition of FuzErmAvPAL for AvPAL expression when in a probiotic host; and

FIG. 2 shows trans-cinnamic acid production by plasmid carried/100-23 cell line in an embodiment of the present invention.

### DETAILED DESCRIPTION OF PREFERRED EMBODIMENTS

One aspect of the present invention pertains to a genetically modified probiotic (GMP) to deliver functional PAL enzyme or other phenylalanine-degrading enzyme to the body of an animal, preferably to the small intestine. In certain embodiments, the probiotic is/has 1) capable of surviving the acid of the stomach and maintaining functionality after exiting the stomach; 2) capable of maintaining functionality and metabolic activity in the small intestine, and expressing the PAL gene or other phenylalanine-degrading enzyme in the intestine; 3) a mouse trophic and human trophic strain; 4) safe for oral use as a human probiotic; 5) capable of being produced in-vitro; and 6) amendable to electropration or another form of transformation.

In some embodiments, the probiotic capable of delivering functional phenylalanine-degrading enzyme, such as PAL enzyme, is useful in the treatment of PKU. If phenylalanine is pulled from the intestinal lumen by probiotics to be degraded by bacterial cells, phe will not enter the blood and will be unable to cause its pathogenic effects in a PKU animal/patient.

In certain embodiments, the probiotic is any of the genera of probiotic bacteria. Examples of probiotics include, but are not limited to, the Lactobacilli and Lactococci genera, which are used in yogurts and probiotic supplements in the USA, and would serve well as a safe delivery system to treat disease. Strain specificity may be selected based on the organism in which the treatment is occurring (i.e. a mouse specific strain may be selected for mouse in-vivo experimentation, while a human specific strain may be selected for human clinical trials and human treatment).

Because amino acid absorption occurs in the small intestine, a probiotic may be selected that is metabolically active in, and preferentially lives in the small intestine. Strains of Lactobacillus reuteri exist for mouse and human, and the microbe adheres to mucus of small intestine in both cases. Finally, a high in vivo retention rate of foreign DNA in this organism can be achieved by using a specific plasmid section in the final shuttle vector. Integration of the desired gene into the chromosome may also be performed if desired.

The enzyme for phenylalanine (phe) degradation may be selected from the enzyme family of Phenylalanine Ammonia Lysases (PALs). PAL enzymes cleave phenylalanine into trans-cinnamic acid and ammonia, both of which are safely and readily cleared from the mammalian body. Unlike other phe catabolizing enzymes, PAL enzymes do not require co-factors or co-enzymes to function. Optimal pH ranges for PAL enzymes are also compatible with proposed physiological pH of the intestine. The multiple potential sources of PAL include plants such as parsley (Petroselinum crispum), eubacteria (Streptomyces maritimus), cyanobacteria (Anabaena variabilis, Nostoc punctiforme) and yeast (Rhodoturula glutinis) or other as yet un-discovered sources.

The selected PAL gene may be codon optimized to allow for successful synthesis (translation) of the enzyme in its probiotic host. This is because each organism has a different codon usage frequency for translating mRNAs into proteins/enzymes, and the PAL gene to be inserted must comply with the host codon usage rather than that of its donor. Each organism typically has multiple codon choices available for each amino acid (AA). Therefore, there are multiple potential DNA sequences that may be used in any particular organism that will result in the same AA sequence while still complying with the probiotic host's codon usage.

Modifications in front of the gene selected for the GMP system may be generated to regulate transcription, translation, and location of the translated PAL enzyme. Additionally, a transcriptional terminator may be added to the end of the gene. Restriction sites may be added as needed throughout the gene. In general, known protocols will be used for DNA amplification, electrophoresis and isolation. In some cases, gel electrophoresis may be conducted in an agarose gel loaded in a “mirror” pattern, such that the same samples are loaded in wells equidistant from an imaginary line running down the center of the gel. After running the gel, the gel will be cut in half along the center of the “mirror”. One half will be stained with Ethidium bromide and visualized. The visualized bands will be measured for distance from the load point, and that measurement will be used to cut out the band on the unstained (Ethidium bromide free) half of the gel. This reduces the likelihood of spontaneous mutation during handling.

Transcription:

A promoter with constitutive expression may be selected and placed in front of the PAL gene in order to drive transcription of the gene. The promoter may be active in vivo, or both in vivo and in vitro. Several examples of good constitutive promoters in Lactobacilli and Lactococci include, but are not limited to: ermB, ldhL, lacA, and sip. It is also possible to place a promoterless PAL gene in tandem behind a gene with a functional promoter as long as the gene with a functional promoter does not contain an intervening termination sequence. If the gene with a promoter does contain a termination sequence, the PAL gene must disrupt the original termination sequence while replicating the termination sequence at the end of PAL.

Translation:

Following the promoter there may be a ribosome binding site (RBS) with an appropriate length of spacer prior to the start codon. Although there are some general similarities of RBS sequences and spacer length amongst prokaryotes, each species has its own preferences. For Lactobacillus and Lactococci, a RBS that is rich in adenine (A) and guanine (G) and is 5-6 bases in length appears to be optimal. The spacer following the RBS and ending at the start codon tends to be between 7 and 13 bases long.

Enzyme Location:

If it becomes desirable to secrete PAL to the cell's exterior, a secretion tag from another enzyme may be added. Examples include, but are not limited to, the secretion tags from the genes Lp_{—}0373, amyL, nlp1, usp45, and mub.

The final gene construct for the GMP system must be introduced into the probiotic in a stable manner. This may be done by placing a PAL gene construct into an existing high retention plasmid or high retention shuttle plasmid (with or without a selection cassette), synthetically creating a high retention plasmid/shuttle plasmid, homologously recombining the PAL gene into the probiotic's chromosome, phage transduction, or usage of a transposable element. A high retention plasmid is designed to be retained in the bacteria for several generations in the absence of selective pressure. If selection is used in the mouse version of this treatment, an antibiotic resistance gene may be used, such as an ampicillin or erythromycin resistance gene. In some embodiments, erythromycin resistance genes C, B, or GT may be used. An E. coli origin of replication to the plasmid may be used, such as the origin in pUC19 (originally from pMB1). In a human version of this treatment, and FDA approved selection cassette would be used, as antibiotic resistance is not acceptable in human probiotics.

After transformation of the probiotic with the gene construct, in vitro phe metabolism by the GMP can be assessed by testing for an increase in absorbance at a wavelength of 280 nm indicating increase in trans-cinnamic acid production.

The genetically modified probiotic “PHEnominal” organism disclosed herein will have the ability to live in the small intestine, where it will break down phenylalanine (phe) prior to its absorption into the blood stream. By preventing excess phenylalanine from entering the blood stream, phenylalanine toxicity will be prevented.

In certain embodiments, the probiotic organism of the present invention will be delivered to an animal or patient as a purified microbe free of any culture media. Purified probiotic organisms may be administered in a pill, powder or drops. These methods of administration have the advantage that a higher quantity of the organism may be delivered than with a culture medium such as yogurt (i.e. the potential quantity of yogurt to be ingested could be much more than would typically be consumed). Moreover, a purified probiotic organism such as the present invention does not present problems with lactose or dairy intolerant patients, or infants who are not ready to eat solid foods. Finally, administration by pill, powder or drops has the advantage that the probiotic organism can be administered with a range of foods, and patients will not develop a dislike of a food eaten every day, as could be the case with probiotic delivered in yogurt.

The appropriate dosage of the GMP system can be calculated by measuring the number of live organisms which must be consumed by an animal to be recovered live from the feces. This number can be combined with survival tests of the probiotic to arrive at an approximate dosage.

The genetically engineered probiotic can be tested as a treatment using an animal model, for example the PAHenu2 mouse on the C57BL6 background with the appropriate controls. Blood collection can be used to assess serum phe throughout the experiment. Tissue collection at the end of experiment will allow for other health assessments.

Shipping costs of the probiotic organism of the present invention in a purified and freeze-dried form will be more cost-effective than shipping of probiotic in a culture medium, such as yogurt, which may need to be refrigerated, and which may weigh significantly more. Moreover, the administration of purified probiotic in pill, powder, or drop form, as in the present invention, may allow a greater degree of dosage tailoring from patient to patient and possibly day to day for the same patient than probiotic delivered in culture media such as yogurt.

Patients with PKU have typically not eaten dairy products due to their high phe content. The sudden addition of regular and potentially high amounts of dairy could cause side effects in these patients, which would again favor the administration of purified microbe in pill, powder or drop form, as described herein.

Probiotics are comparatively inexpensive to produce. Because the proposed genetically modified probiotic would be classified as a “drug”, it may be more likely to be accepted by insurance companies as a treatment they cover (when compared to synthetic foods). Using a phenylalanine degrading probiotic will likely result in higher treatment compliance, as it will allow patients to maintain a more normal diet and life style and not one in which they would have to ingest distasteful foods. As treatment with “PHEmoninal” would require oral administration, it would not require injections as with the PEG-PAL treatment (see above).

### Example 1

### Construction of a GMP System—PHEnominal

The species Lactobacillus reuteri is used as the probiotic. Strains of this organism are found in the small intestine of most higher animals including humans and mice. Strains that are human and mouse specific have been identified and studied. For the proof of principal experiments in-vitro and the mouse in-vivo work, we will use Lactobacillus reuteri 100-23C. This strain has no indigenous plasmids and is capable of colonizing the small intestine of Lactobacillus free mice. When progressing to human clinical trials, a human specific strain of L. reuteri or an alternative probiotic as approved by the FDA will be used.

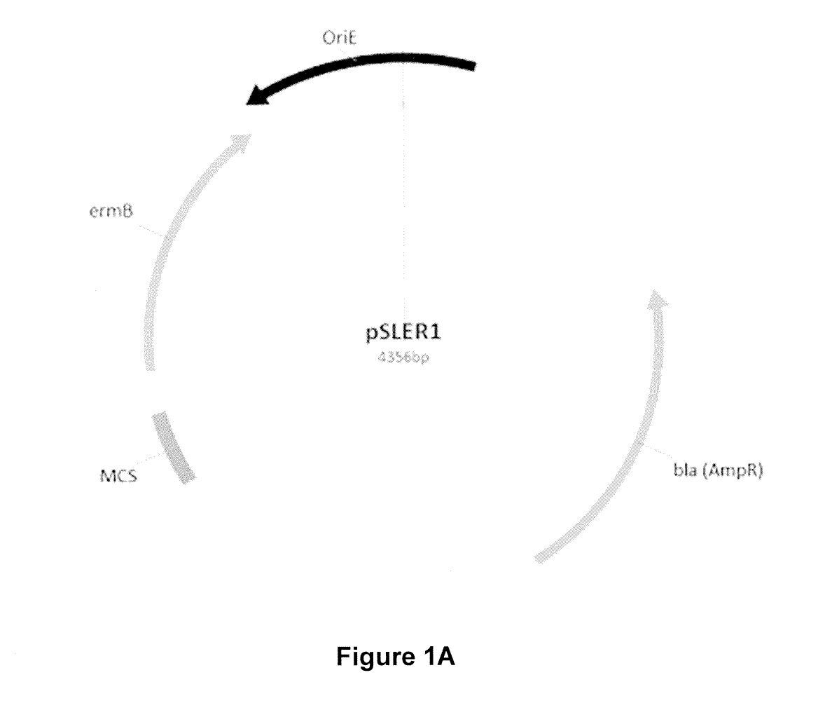

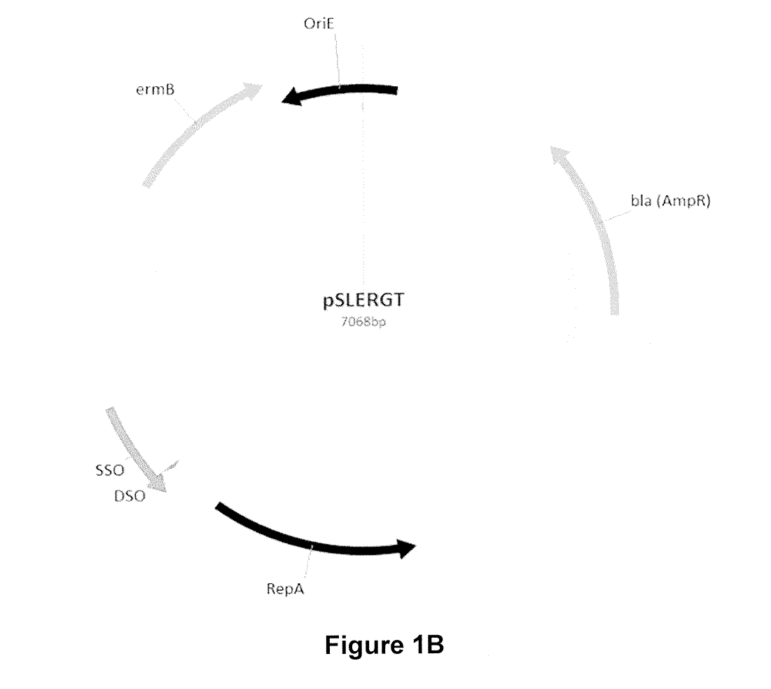

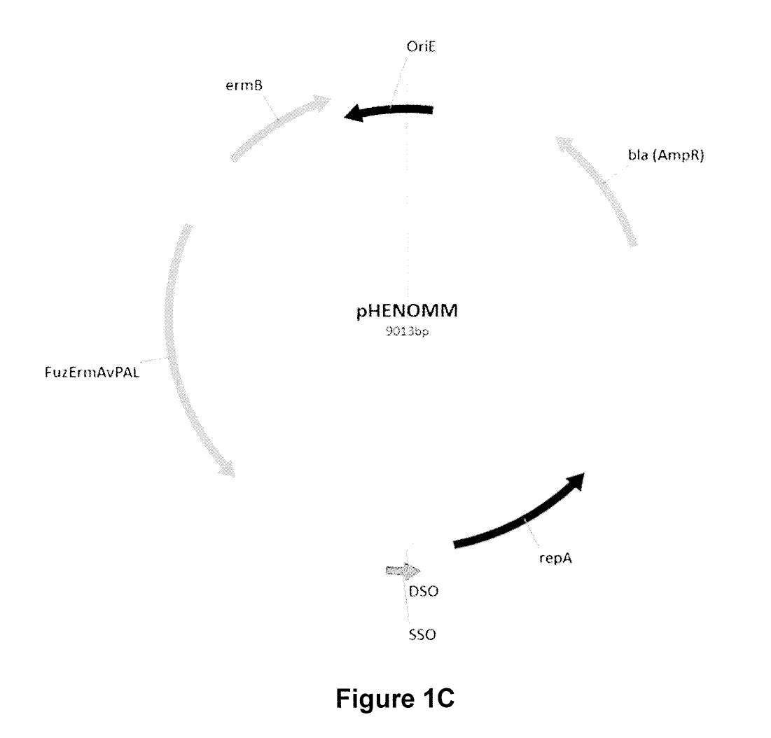

The PAL enzyme to be used is from the cyanobacterium Anabaena variabilis ATCC 29413, referred to hereinafter as AvPAL. The sequence for this gene will be codon optimized to match codon usage for the Lactobacillus reuteri species as a whole. The AvPAL front sequence (SEQ ID NO: 1) will be placed in front of the codon optimized gene to act as an RBS and spacer sequence. At the end of the gene, the AvPAL end sequence (SEQ ID NO: 2) will be added to act as a transcriptional terminator. This promoterless AvPAL construct (AvPAL plus the above detailed additional sequences) will be synthesized (BioBasic Inc., Ontario, Canada). The complete AvPAL is shown in SEQ ID NO: 3. All sequences not otherwise noted are right facing and in the standard left to right orientation. Plasmids pSLER1, pSLERGT, pHENOMM, and pHENOMM-sec are all left facing and are read right to left. In all plasmids it should be noted that the ermB gene is in the opposite orientation of the plasmid, and this gene is right facing reading left to right (as depicted by plasmid maps in FIGS. 1A, 1B, and 1C).

A promoter is attached to the AvPAL construct in order to create a functional AvPAL construct. The ermB promoter region with 10 additional bases inserted (SEQ ID NO: 4) was selected. This process of fusing the synthesized synthetic ermB promoter to AvPAL created the final gene product of FuzErmAvPAL (SEQ ID NO:15) and was performed by BioBasic Inc, Ontario, Canada. Additionally, the secretion tag of Lp_{—}0373 (SEQ ID NO: 5) may be inserted between the ermB promoter and the start codon of the AvPAL gene for secFuzErmAvPAL (SEQ ID NO:6). Once the full construct was created, BioBasic ligated FuzErmAvPAL and secFuzErmAvPAL separately into our synthetically created shuttle vector pSLERGT.

Once FuzErmAvPAL and secFuzErmAvPAL have been synthesised, they must become part of a functional Lactobacillus reuteri shuttle plasmid. The shuttle plasmid to be used, pSLERGT (SEQ ID NO: 7), was created specifically for the mouse project by using an antibiotic resistance gene such as erythromycin resistance genes C, B, or GT as the selection cassette, the pGT232 fragment from pNCKH103 (SEQ ID NO: 8) for stability in-vitro and in-vivo in a Lactobacillus reuteri host even in the absence of selection, and E. coli origin of replication for shuttle function. The E. coli vector pSLER1 (SEQ ID NO: 9) contains the elements ermB (erythromycin resistance gene B) and E. coli origin of replication and will be used as the back bone of shuttle vector creation because of these properties. For a human safe plasmid, a selectable gene such as heavy metal resistance or alanine racemase (for complementation) may be used instead of antibiotic resistance. It is also possible in mouse or human safe version to integrate the desired functional AvPAL construct directly into the chromosomal DNA.

To create the desired shuttle vector of pSLERGT, plasmid DNA of pNCKH103 was harvested from 100-23 cells to allow extension PCR of the pGT232 fragment. The reaction used a forward primer for pGT232 amplification (SEQ ID NO: 10) and a reverse primer for pGT232 amplification (SEQ ID NO: 11). The following reaction mixture of New England Biolabs Q5 High Fidelity DNA polymerase was used for a 30 μl volume per reaction tube.

| 10x Q5 polymerase reaction Buffer | 6 | μl |
|---|---|---|
| pNCKH103 DNA from L.r. 100-23 | 1.5 μl of 15 ng/μl total DNA* | |
| Forward Primer | 1.5 | μl |
| Reverse Primer | 1.5 | μl |
| dNTP 10 mM | 2.4 | μl |
| Water | 18.6 | μl |
| Q5 DNA polymerase | 0.25 | μl |

*Total DNA, rather than plasmid DNA, is reported due to the dirty quality of DNA extracted from L.r. 100-23 cells.

The thermocycler conditions used for amplification were

| Step | ° C. | Time (minutes:seconds) |
|---|---|---|
| 1 | 98.0 | 0:30 |
| 2 | 98.0 | 0:10 |
| 3 | 57.0 | 0:12 |
| 4 | 72.0 | 1:30 |
| 5 | go to step 2, x4 | |
| 6 | 98.0 | 0:10 |
| 7 | 65.0 | 0:12 |
| 8 | 72.0 | 1:30 |
| 9 | Go to step 6, x29 | |
| 10 | 72 | 2:00 |
| 11 | 4 | ∞ |

The resulting PCR products were then run on a 0.8% agarose gel with 0.5% TBE. Gels run to optimize the reaction conditions were run with ethidium bromide incorporated into the gel. Gels for harvesting DNA for plasmid creation were run in the absence of Ethidium Bromide. For harvesting, the gel was loaded such that the first lane was a DNA ladder, and as many remaining lanes as could be filled contained PCR product. Once the Coomassie blue band reached the end of the gel, electrophoresis was stopped. The gel was cut into 2 sections, so that lanes 1 and 2 were together and the remaining lanes in the second portion. The gel segment with lanes 1 and 2 was stained in a 0.5% TBE ethidium bromide bath in a rocker for 20 minutes to visualize the PCR products with a Fotodyne Incorporated Imager and Foto/Analist PC Image software. The desired pGT232 fragment was excised from this stained portion, and revisualized to confirm appropriate cutting. This stained gel was then used as a guide to cut the appropriate bands from the lanes in the unstained portion of the gel. The bands that were never exposed to ethidium bromide or UV light were used in the UltraClean® GelSpin® DNA Extraction Kit (MoBio Laboratories, Inc., Carlsbad, Calif.). DNA post gel purification was quantitated with a Nanodrop photospectrometer.

The PCR created pGT232 fragment was cut using NcoI FastDigest (Thermoscientific) for one sticky and one blunt end. The E. coli vector/backbone for insertion of the PCR product, pSLER1, was cut using FastDigest enzymes NcoI and SmaI resulting in a sticky blunt vector. Reactants for the reactions as follows

pGT232fragment digest
4 μl of pGT232fragment at 17 ng/μl
0.5 μl 10× ThermoScientific FastDigest buffer
0.5 μl Thermoscientific FastDigest NcoI enzyme
pSLER1 digest
1.3 μl pSLER1 at 750 ng/μl
14.7 μl water
2 μl 10× ThermoScientific FastDigest buffer
1 μl Thermoscientific FastDigest NcoI enzyme
1 μl Thermoscientific FastDigest SmaI enzyme

The reactions were incubated in a 37° C. water bath for 1.5 hours.

Small DNA fragments created by the digests and the enzymes used were removed from the desired DNA using the solutions protocol of the UltraClean® GelSpin® DNA Extraction Kit (MoBio Laboratories, Inc., Carlsbad, Calif.) and eluted into molecular water. The resulting DNA concentration was again determined by Nanodrop.

Ligation of the digested, PCR created pGT232 fragment into the sticky blunt pSLER1 vector was done using the T4 DNA ligase from Thermoscientific per the following reaction.

Ligation of pGT232fragment into pSLER1 for creation of pSLERGT
2 μl 10× T4 DNA ligase Buffer by ThermoScientific

### 2 μl 50% PEG 4000

1 μl pSLER1 digested at 21 ng/μl
5.5 μl pGT232 fragment digested at 6 ng/μl
8.5 μl water

Ligation reaction was at room temperature for 60 min. 5 μl of the ligation reaction was added to 50 μl of chemically competent Top10 DH5α E. coli from Life Technologies for transformation. Transformation was performed as described by the manufacterer. Transformation reaction was plated onto LB agar containing 50 μg/mL of ampicillin.

Colonies from the plates were sub-cultured in TB dry containing 300 μg/mL of erythromycin. These liquid cultures were used for the UltraClean 6 minute mini plasmid prep kit (MoBio). Plasmid samples were digested with ThermoScientific FastDigest NcoI and SalI, and the resulting digests were run on a 0.8% agarose 0.5% TBE with ethidium bromide gel to detect clones containing a plasmid with the pGT232 fragment insert.

The clone with successful insertion, the desired pSLERGT, was sent to BioBasic for sequencing. Once the sequencing verified the insert contained no mutations to alter function of the pGT232 fragment (SEQ ID NO:12), pSLERGT was transformed into 100-23C cells to verify functionality of the pGT232 segment in-vitro. pSLERGT successfully transformed 100-23 cells in addition to the E. coli, proving it a functional shuttle vector.

The reactants and conditions for electotrotransforma ion of the 100-23C cells are as described in M. A. McCONNELL et al., “Transfer of Plasmid pAMβ1 Between Members of the Normal Microflora Inhabiting the Murine Digestive Tract and Modification of the Plasmid in a Lactobacillus reuteri Host.”

Briefly, a Gene Pulser™′ apparatus (Bio-Rad Laboratories, Richmond, Calif.) will be used for all electroporation experiments described in this study. Recipient cells from overnight cultures at 37° C. in MRS broth will be used to inoculate MRS broth to an optical density of 0.06 at 600 nm (OD). Cultures will be incubated at 37° C. until an OD 600 of 0.8-1.0 is attained. The cells will be harvested by centrifugation, washed twice in electroporation buffer, and then resuspended in electroporation buffer at 1/20th of the original culture volume. Electroporation buffer consists of 952 mM sucrose-3.5 mM MgCl₂ at pH 7.2 that has been filter sterilized.

Plasmid DNA, ≧1 μg, will be added to 0.4 ml of cells. This mixture is then placed in chilled sterile electroporation cuvettes (0.2 cm inter-electrode gap) and held on ice for 5 min. Following the application of a high-voltage electric pulse, voltage of 12,500 V/cm, 200 ohm parallel resistance, 25 μFD capacitance, the DNA-cell mixture is added to 10 mL of pre-warmed non-selective media for a 3 hour recovery period at 37° C. After recovery, cells are harvested by centrifugation and resuspended in 1 mL of media for plating. Approximately 50 μl of this solution will be plated onto selective media with 5 μg/mL of antibiotic. Diluted aliquots will also be plated on media without antibiotic to ensure cells remained viable throughout the procedure regardless of their plasmid uptake. After incubation of the plates in an anaerobe jar at 37° C. for approximately 40 h, colonies will be counted/utilized.

Colonies that grow will be positive for the plasmid, and these colonies will be grown in an MRS broth culture in the anaerobe jar with its gas pack components at 37° C. over night. Growth from these MRS overnight cultures will be our stock culture, and as such they will be prepared by using a 1:1 ratio of culture to 10% sterile skim milk and saved by freezing at −80° C.

The final shuttle vector pSLERGT was sent to BioBasic Inc. as mentioned above for ligation of the final FuzErmAvPAL or secFuzErmAvPAL into pSLERGT to create the therapeutic test vectors pHENOMM (SEQ ID NO:13) and pHENOMM-sec (SEQ ID NO:14) respectively.

The resulting plasmid (pHENOMM or pHENOMH) carried by L. reuteri are considered an embodiment of the present invention. Probiotic carrying pHENOMM or pHENOMH will be called PHEnominal. Strain specificity of the probiotic and plasmid used denotes its human or mouse usage. Stock vials of the appropriate strain will be thawed and cultured in MRS broth and anaerobic conditions at 37° C. overnight for in-vitro and in-vivo experimentation. A single vial of stock culture may be used for a long period of study by subculturing the most recent growth of PHEnominal.

### Example 2

### Administration and Use of the Oral PHEnominal to Treat PKU

In certain embodiments, the genetically engineered probiotic to treat PKU described herein may be orally administered with food in mice and humans. Survival of the genetically modified probiotic to treat PKU (“PHEnominal”) will be greater if it is ingested with food and this will be enhanced further if dairy is ingested concomitant with food. Modes of administration of the oral treatment are varied and may be in pill form, or powder/liquid form. The most appropriate form would be selected for the age and life stage of the patient (e.g. toddlers and infants often resist swallowing pills).

A patient may be provided with access to a phenylalanine monitor to determine levels of phe in the blood. As an essential amino acid, the patient must ensure they are not taking too much of the genetically engineered probiotic. Such phenylalanine monitors for home use are currently in clinical trials, though more expensive versions already exist (approximately $40,000.00 for the currently available machine).

### Example 2

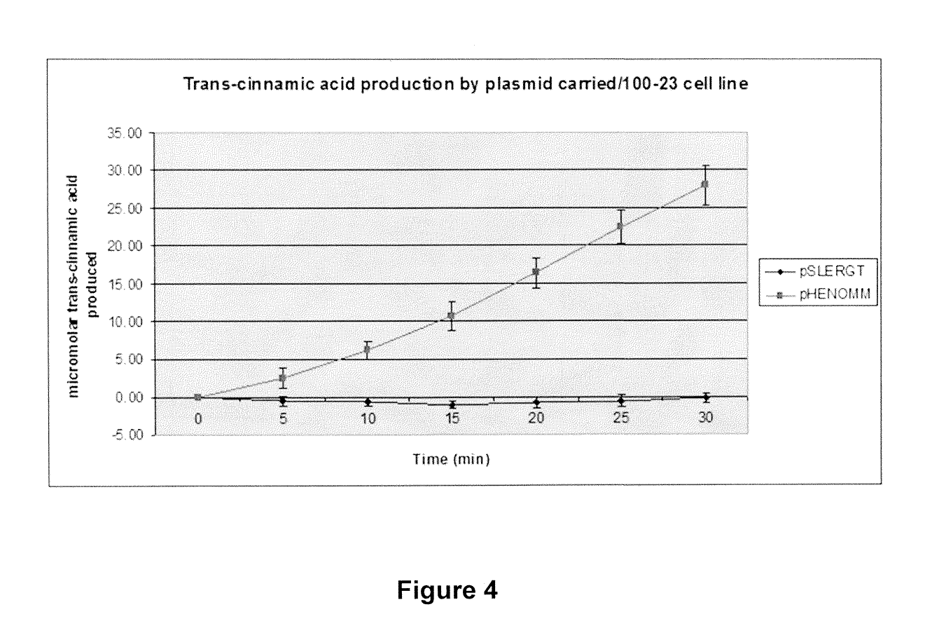

At this time, testing of mouse PHEnominal has been performed to demonstrate functional AvPAL enzyme within the cells. Lysate from 100-23 cells carrying pHENOMM (bearing the FuzErmAvPAL gene, non-secreted) plasmid contain far higher production of trans-cinnamic acid than lysate from cells carrying the empty vector pSLERGT. A representative set of results are seen in FIG. 2, and the data retains this trend over multiple experimental replicates. Values depicted indicate the amount of increase in absorbance readings at 280 nm, the absorbance point for trans-cinnamic acid/trans-cinnamate, in lysates given phosphate buffered saline with phenylalanine over the amount produced by cells only given phosphate buffered saline alone. Subtracting the values for phosphate buffered saline alone from values measured in the presence of phenylalanine accounts for any increase to the absorbance created by other reactions. These results demonstrate Lactobacillus reuteri cells are capable of surviving while producing functional AvPAL enzyme.

### REFERENCES CITED

The following references, to the extent that they provide exemplary procedural or other details supplementary to those set forth herein, are specifically incorporated herein by reference.

### Non-Patent Literature

Bourget, L. Chang, T. Phenylalanine ammonia-lyase immobilized in semipermeable mircrocapsules for enzyme replacement in phenylketonuria. Federation of European Biochemical Societies, 1985 vol 180 number 1 pp 5-9.
Bourget L, Chang T M S. Biochim Biophys Acta. 1986; 883:432-438
Safos S, Chang T M S. Artif Cells Blood Substit Immobil Biotechnol. 1995; 23:681-692
Sarkissian C N, Lee K C, Danagher P, Leung R, Fuller M A, Scriver C R. Am J Hum Genet Suppl. 1996; 59:1183. (abstr.).
Sarkissian C N, Shao Z, Blain F, Peevers R, Su H, Fuller M A, Scriver C R. Am J Hum Genet Suppl. 1997; 61:182. (abstr.).
Liu et al. Study on a Novel Strategy to Treatment of Phenylketonuria. Artif cells, Blood Substit Immobil Biotechnol, 2002, 30(4), 243-257.
http://www.pkunsw.org.au/research-blog-full-article
M. A. McCONNELL et al., “Transfer of Plasmid pAMβ1 Between Members of the Normal Microflora Inhabiting the Murine Digestive Tract and Modification of the Plasmid in a Lactobacillus reuteri Host”

What is claimed:

1. A composition for treating phenylketonuria, comprising:
a) a probiotic bacterium; and
b) a gene capable of expressing a functional enzyme for phenylalanine degradation by the probiotic bacterium.

2. The composition of claim 1, wherein the probiotic bacterium is selected from the Lactobacilli and Lactococci genera.

3. The composition of claim 1, wherein the probiotic bacterium is Lactobacillus reuteri.

4. The composition of claim 1, wherein the gene capable of expressing a functional enzyme for phenylalanine degradation is selected from the enzyme family of phenylalanine ammonia-lyases.

5. The composition of claim 4, wherein the gene capable of expressing a functional enzyme for phenylalanine degradation is derived from parsley (Petroselinum crispum), eubacteria (Streptomyces maritimus), cyanobacteria (Anabaena variabilis, Nostoc punctiforme) or yeast (Rhodoturula glutinis).

6. The composition of claim 1, wherein the gene capable of expressing a functional enzyme for phenylalanine degradation is cyanobacterium Anabaena variabilis ATCC 29413.

7. The composition of claim 1, wherein the gene capable of expressing a functional enzyme for phenylalanine degradation is codon-optimzed.

8. The composition of claim 1, wherein the gene capable of expressing a functional enzyme for phenylalanine degradation includes a promoter.

9. The composition of claim 8, wherein the promoter exhibits constitutive expression.

10. The composition of claim 1, wherein the gene capable of expressing a functional enzyme for phenylalanine degradation includes a ribosome binding site.

11. The composition of claim 1, wherein the gene capable of expressing a functional enzyme for phenylalanine degradation includes a secretion sequence.

12. The composition of claim 1, wherein the gene is capable of expressing a functional enzyme for phenylalanine degradation is located in a high retention plasmid within the probiotic.

13. The composition of claim 1, wherein the gene capable of expressing functional enzyme for phenylalanine degradation comprises SEQ ID NO:3, or a functional variant thereof.

14. The composition of claim 1, wherein the gene capable of expressing functional enzyme for phenylalanine degradation comprises SEQ ID NO:13, or a functional variant thereof.

15. The composition of claim 1, wherein the gene capable of expressing functional enzyme for phenylalanine degradation comprises SEQ ID NO:14, or a functional variant thereof.

16. A method for treating phenylketonuria, comprising the step of:
administering the composition of claim 1 to a patient.

17. The method of claim 16, wherein administration is oral.


SEQUENCE LISTING (15 sequences)

SEQ ID NO: 1 - DNA, 14 bp, Artificial Sequence
  Sequence to be added to to front of codon optimized AvPAL sequence.
aggagtaggtataa

SEQ ID NO: 2 - DNA, 56 bp, Artificial Sequence
  Sequence to be added to the end of AvPAL sequence.
tgataagttaaggggtgcataaactgcatcccttaacttatcaaaaaaaagtcgac

SEQ ID NO: 3 - DNA, 1777 bp, Artificial Sequence
  Synthesized AvPAL 5′ to 3′
aggagtaggtataaatgaagactttatctcaagcacaatctaagactagttctcaacaattctcatttactggaaactcaagtgctaatgttatcattggtaatcaaaagcttactattaacgatgttgcacgggttgcacgcaatggtactttagttagtttaactaacaacactgacattttacaaggcattcaagctagttgcgactatatcaataacgctgttgaatcaggagaaccaatttatggtgttacttcaggattcggtggaatggcaaacgtcgctattagtcgtgaacaagcttctgaacttcaaactaatcttgtttggtttcttaaaacaggagctggtaataaacttccattagcagatgttcgtgctgctatgttattacgtgcaaattcacacatgcgtggcgcttctggtattcgtcttgaattaattaagcgtatggaaattttccttaacgccggtgttacaccttatgtttacgaattcggatcaattggtgcaagtggagatcttgttcctttatcttatattacaggttctttaattggtttggatccatcattcaaagttgattttaatggtaaagaaatggatgccccaacagctcttcgacaattaaatttatcaccacttacattacttccaaaagaaggtcttgctatgatgaatggtacatcagttatgacgggaattgctgctaattgcgtttatgatactcaaattcttactgctattgccatgggtgttcatgccttagatattcaagcacttaatggcacaaatcaaagtttccacccattcattcacaattctaaacctcacccaggtcaactttgggctgctgatcaaatgatttctcttttagctaatagtcaacttgttcgcgacgaattagatggtaagcatgattatcgtgatcatgaacttattcaagaccgctatagtttacgttgcttaccacaatatttgggtcctattgttgatggaatttcacaaattgctaagcaaattgaaatcgaaattaatagtgtcactgacaatccattaattgacgttgataaccaagcctcataccatggcggtaactttttgggtcaatatgttggcatgggcatggaccacttacgttactacattggtcttcttgcaaagcatttggatgtccaaattgcacttttggccagtccagaattcagtaacggacttccaccatcattattgggaaaccgggaacgaaaggtaaacatgggtcttaagggtttgcaaatttgtggcaactcaattatgccattgttgactttttacggaaattcaattgctgatcgctttcctacacacgccgaacaatttaaccaaaatattaactctcaaggatacacttctgctacgttagcacgacggtctgttgatatttttcaaaactacgtagctattgcattaatgtttggagtacaagctgttgatttacggacttacaaaaagacgggccattatgatgctcgtgcttgtttaagtccagctacagaacgtctttattcagcagtacgtcatgttgtcggccaaaaacctacatcagatcggccatatatttggaacgataacgaacaaggacttgacgaacatattgctcgtatttctgctgatatcgctgctggtggtgtaattgttcaagccgtacaagatatccttccttgtttacactaataatgataagttaaggggtgcataaactgcatcccttaacttatcaaaaaaaagtcgac

SEQ ID NO: 4 - DNA, 186 bp, Artificial Sequence
  Synthetic ErmB promoter sequence
gcatgctagctatagttctagaggatacctggttgattaacgttagcctggctacgtatactcctggaagtattaatagacgacctaggatgcatatgttcaagagtgtgttgatagtgcagtatcttaaaattttgtataataggaattgaagttaaattagatgctaaaaatttgtaattaaga

SEQ ID NO: 5 - DNA, 129 bp, Artificial Sequence
  Secretion tag sequence
atgtatactgaaaatacaggtaaacatcaccgtaacggacttcctgtgtggctattaccattattggtcgttattagtttttggggcgtaagccaaaatattatggttgttgatgcttcttcagttgat

SEQ ID NO: 6 - DNA, 2092 bp, Artificial Sequence
  Full secFuzErmAvPAL sequence
gcatgctagctatagttctagaggatacctggttgattaacgttagcctggctacgtatactcctggaagtattaatagacgacctaggatgcatatgttcaagagtgtgttgatagtgcagtatcttaaaattttgtataataggaattgaagttaaattagatgctaaaaatttgtaattaagaaggagtaggtataaatgtatactgaaaatacaggtaaacatcaccgtaacggacttcctgtgtggctattaccattattggtcgttattagtttttggggcgtaagccaaaatattatggttgttgatgcttcttcagttgatatgaagactttatctcaagcacaatctaagactagttctcaacaattctcatttactggaaactcaagtgctaatgttatcattggtaatcaaaagcttactattaacgatgttgcacgggttgcacgcaatggtactttagttagtttaactaacaacactgacattttacaaggcattcaagctagttgcgactatatcaataacgctgttgaatcaggagaaccaatttatggtgttacttcaggattcggtggaatggcaaacgtcgctattagtcgtgaacaagcttctgaacttcaaactaatcttgtttggtttcttaaaacaggagctggtaataaacttccattagcagatgttcgtgctgctatgttattacgtgcaaattcacacatgcgtggcgcttctggtattcgtcttgaattaattaagcgtatggaaattttccttaacgccggtgttacaccttatgtttacgaattcggatcaattggtgcaagtggagatcttgttcctttatcttatattacaggttctttaattggtttggatccatcattcaaagttgattttaatggtaaagaaatggatgccccaacagctcttcgacaattaaatttatcaccacttacattacttccaaaagaaggtcttgctatgatgaatggtacatcagttatgacgggaattgctgctaattgcgtttatgatactcaaattcttactgctattgccatgggtgttcatgccttagatattcaagcacttaatggcacaaatcaaagtttccacccattcattcacaattctaaacctcacccaggtcaactttgggctgctgatcaaatgatttctcttttagctaatagtcaacttgttcgcgacgaattagatggtaagcatgattatcgtgatcatgaacttattcaagaccgctatagtttacgttgcttaccacaatatttgggtcctattgttgatggaatttcacaaattgctaagcaaattgaaatcgaaattaatagtgtcactgacaatccattaattgacgttgataaccaagcctcataccatggcggtaactttttgggtcaatatgttggcatgggcatggaccacttacgttactacattggtcttcttgcaaagcatttggatgtccaaattgcacttttggccagtccagaattcagtaacggacttccaccatcattattgggaaaccgggaacgaaaggtaaacatgggtcttaagggtttgcaaatttgtggcaactcaattatgccattgttgactttttacggaaattcaattgctgatcgctttcctacacacgccgaacaatttaaccaaaatattaactctcaaggatacacttctgctacgttagcacgacggtctgttgatatttttcaaaactacgtagctattgcattaatgtttggagtacaagctgttgatttacggacttacaaaaagacgggccattatgatgctcgtgcttgtttaagtccagctacagaacgtctttattcagcagtacgtcatgttgtcggccaaaaacctacatcagatcggccatatatttggaacgataacgaacaaggacttgacgaacatattgctcgtatttctgctgatatcgctgctggtggtgtaattgttcaagccgtacaagatatccttccttgtttacactaataatgataagttaaggggtgcataaactgcatcccttaacttatcaaaaaaaagtcgac

SEQ ID NO: 7 - DNA, 7068 bp, Artificial Sequence
  pSLERGT sequence
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaatggccatggaggatttattctctccgcagttttgagcttaatcgtctcattaactactgcacttttctccgattggcttcgtcggagaaaataatctaacggcatgaagcgcaaattaaaagccactcaactatgggcctagttgggtggcttttttgcaaccattacggttttacttctctataagaattatagcatcaatcttcatcattagccaactcaatagaagctcgatatagatcatgttgagctttatccaagctatctaaaagttcgccataaatatcaatatttttcttttcagcaaccaattttttcaaatcttgaagattatcagcaatctgttctgccataactccatagatttcagcttgtttcttcatattgtcaattgtcttaatatcttgtaactccattcctatcactcccatcttacatagttacttaccttagaattccaacgatatatcacagtatctacaacatcatctttttctttttcgtcatccacattaattagatcatcttctttttcgtccagcaataactcatgatgaatctcttttagtaatcccgcaaagcttaattgtcgagttccagccagagcatgctcgagctcgtcaacaacaactaaatcgcctttctcatcatccgtaatataatcataatctttaacttgatatttacttacttcttttgccgaagcaatcaaactattatctttttttgacgcccgaattttcttgatattaacaatcggcttgtagtctaatttccttgctcgcttccataattgtgaccaattctcttgggtaagataatgacccttagaaaaataactagatttcaccattaacaaaatatgtacatgttgatggaaagttagactatctcgattgaccgttatttctgtagatcgcacataaccaagaagatctttttttacatctttatattgaaaaagccgataaattgaacgattcatcgaagtaagattttttctaagctcgcccaactttgagctttcctcagttaatgtcaagaacagaaatatagcactaggatcctttttatatgctgtatctaaaatttgacgcaactcataagaatttttcatcgatcggcgccagctacataatggacaaagtctcgaatgacaaaaccacgtttgatataactttaaaccattatcagttttcgcaaattgaagaacttctccgcactttgaaacgttatgagcttttttaaaatttaaaacctccagatattgggaataagttaagttagcaagttttttctgcttccacggtctaactttcccattactagaggtatcttttagaatttcttttttatacatttcttatacctctaactgcaaaaactacaagcagtcagagttagttatcattgatatattcctaatgacatattataatagggttcgaagttaataacgataactaactcatgcatcgacggccaaatcgaacaactgcatgagttttttatttaattcttacaatgcttattatacaccaaaaagtgttatattttaaaatcctaataaacccagtcatatcaagggtttaacccaatttcaaaaaatggcgttgtttctatatgtatcaagataagaagaaactcgcttacagcgagtttctaaataggggtttcacccctaaaaccctgaccagctgagccagctggaccgcatttcccctcgccgggcgggcgtaaagcggaaaaaatgacttttttctcgctttcgctcaactagggtcttcccgtctcaccatgtcaagcgctgcccttccgctacgctccaggcgtttgtcgcttgacactcacctactaacagcgggcttcgccaccagtctactatctgcaacacggcaacaagattaatcagaatatgcattgaagcagtgcgctccgctccttgtcttgcgacaaagcgagcgcactgcttcaatgcttttttattttctgaattccataactcgataattagaaaaagttagtccgttagaaacgtctgccacgccgaggaagtcattccagtttgaccactcaaatcatcagcgacatcattgagataatcaatcagcaattttctagtttctatctgctttggaatttttaccacagccaaaattgagtcccacttagcaatcacaaaagattttctgacggctttattagccttttccgcactaatatttttcgctttatctcgtttattttgatcggtcgatggtatccgatcgactgttgttacagcatttgacgcaggttgctagataaacgactagaccgaataatttttattccaactatttttgcaaccaaaaaaatcacaaaataatcaaccaataaaacgataactaacttcttgctacatccgattaatttttctccagcctgccagaaatcggcccaaatattgaaatattgaggaaattttgggctgaaaaaaacattaattttttcggcaaacaagccaacacagccaagcagaaaagctaagaaaataagttccagccaattaaaaaatcgccacgaaagagaaaaaaatcggtcaaaataaatcgaacttgatttcattttctagcccccgaagtcttcgataaacggcgccataaaaggaactacacggttttccggttgagtacttgcaagccctaaccccttagaaaagccgtcatgattaacaactgttgtcgactcagctagggccctatatatggatccaattgcaatgatcatcatgacagatctgcgcgcgatcgatatcagcgctttaaatttgcgcatgctagctatagttctagaggtaccggttgttaacgttagccggctacgtatactccggaatattaataggcctaggatgcatatgcttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagataaattattaggtatactactgacagcttccaagcatatggcggccgcctgcagctggcgccatcgatacgcgtacgtcgcgaccgcggacatgtacagagctcgagaagtactagtggccacgtgggccgtgcaccttaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 8 - DNA, 2764 bp, Artificial Sequence
  Predicted pGT232 from PCR of pNCKH103
tagctgagtcgacaacagttgttaatcatgacggcttttctaaggggttagggcttcaagtactcaaccggaaaaccgtgtagttccttttatggcgccgtttatcgaagacttcgggggctagaaaatgaaatcaagttcgatttattttgaccgatttttttctctttcgtggcgattttttaattggctggaacttattttcttagcttttctgcttggctgtgttggcttgtttgccgaaaaaattaatgtttttttcagcccaaaatttcctcaatatttcaatatttgggccgatttctggcaggctggagaaaaattaatcggatgtagcaagaagttagttatcgttttattggttgattattttgtgatttttttggttgcaaaaatagttggaataaaaattattcggtctagtcgtttatctagcaacctgcgtcaaatgctgttaacaacagtcgatcggataccatcgaccgatcaaaataaacgagataaagcgaaaaatattagtgcggaaaaggctaataaagccgtcagaaaatcttttgtgattgctaagtgggactcaattttggctgtggtaaaaattccaaagcagatagaaactagaaaattgctgattgattatctcaatgatgtcgctgatgatttgagtggtcaaactggaatgacttcctcggcgtggcagacgtttctaacggactaactttttctaattatcgagttatggaattcagaaaataaaaaagcattgaagcagtgcgctcgctttgtcgcaagacaaggagcggagcgcactgcttcaatgcatattctgattaatcttgttgccgtgttgcagatagtagactggtgcgaagcccgctgttagtaggtgagtgtcaagcgacacaacgcctggagcgtgagcggaagggcagcgcttgacatggtgagacgggaagaccctagttgagcgaaagcgagaaaaaagtcattttttccgctttagcccgcccggcgaggggaaatgcggtccagctgggtcagggttttagggggtgaaacatccttagaaactcgctgtaagcgagtttcttcttatcttgatacatatagaaacaacgccattttttgaaattgggttaaacccttgatatgactgggtttattaggattttaaaatataacactttttggtgtataataagcattgtaagaattaaataaaaaactcatgcagttgttcgatttggccgtcgatgcatcagttagttatcgttattaacttcgaaccctattataatatgtcattaggaatatatcaatgataactaactctgactgcttgtagtttttgcagttagaggtataagaaatgtataaaaaagaaattctaaaagatacctctagtaatgggaaagttagaccgtggaagcagaaaaaacttgctaacttaacttattcccaatatctggaggttttaaattttaaaaaagctcataacgtttcaaagtgcggagaagttcttcaatttgcgaaaactgataatggtttaaagttatatcaaacgtggttttgtcattcgagactttgtccattatgtagctggcgccgatcgatgaaaaattcttatgagttgcgtcaaattttagatacagcatataaaaaggatcctagtgctatatttctcttcttgacattaactgaggaaagctcaaagttgggcgagcttagaaaaaatcttacttcgatgaatcgttcaatttatcggctttttcaatataaagatgtaaaaaaagatcttcttggttatgtgcgatctacagaaataacggtcaatcgagatagtctaactttccatcaacatgtacatattttgttaatggtgaaatctagttatttttctaagggtcattatcttacccaagagaattggtcacaattatggaagcgagcaaggaaattagactacaagccgattgttaatatcaagaaaattcgggcgtcaaaaaaagataatagtttgattgcttcggcaaaagaagtaagtaaatatcaagttaaagattatgattatattacggatgatgagaaaggcgatttagttgttgttgacgagctcgagcatgctctggctggaactcgacaattaagctttgcgggattactaaaagagattcatcatgagttattgctggacgaaaaagaagatgatctaattaatgtggatgacgaaaaagaaaaagatgatgttgtagatactgtgatatatcgttggaattctaaggtaagtaactatgtaagatgggagtgataggaatggagttacaagatattaagacaattgacaatatgaagaaacaagctgaaatctatggagttatgccagaacagattgctgataatcttcaagatttgaaaaaattggttgctgaaaagaaaaatattgatatttatggcgaacttttagatagcttggataaagctcaacatgatctatatcgagcttctattgagttggctaatgatgaagattgatgctataattcttatagagaagtaaaaccgtaatggttgcaaaaaagccacccaactaggcccatagttgagtggcttttaatttgcgcttcatgccgttagattattttctccgacgaagccaatcggagaaaagtgcagtagttaatgagacgattaagctcaaaactgcggagagaataaatcctccatggtttcttagacggagagaataaatcctccatggtttcttaga

SEQ ID NO: 9 - DNA, 4356 bp, Artificial Sequence
  pSLER1 sequence
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaatggccatgggacgtcgacctgaggtaattataacccgggccctatatatggatccaattgcaatgatcatcatgacagatctgcgcgcgatcgatatcagcgctttaaatttgcgcatgctagctatagttctagaggtaccggttgttaacgttagccggctacgtatactccggaatattaataggcctaggatgcatatgttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagataaattattaggtatactactgacagcttccaagcatatggcggccgcctgcagctggcgccatcgatacgcgtacgtcgcgaccgcggacatgtacagagctcgagaagtactagtggccacgtgggccgtgcaccttaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 10 - DNA, 25 bp, Artificial Sequence
  Forward primer pGT232 amplification reaction.
tagctgagtcgacaacagttgttaa

SEQ ID NO: 11 - DNA, 30 bp, Artificial Sequence
  Reverse primer pGT232 amplification reaction.
tctaagaaaccatggaggatttattctctc

SEQ ID NO: 12 - DNA, 3054 bp, Artificial Sequence
  Sequenced pGT232 inserted into pSLER1 for pSLERGT
ccatggaggatttattctctccgcagttttgagcttaatcgtctcattaactactgcacttttctccgattggcttcgtcggagaaaataatctaacggcatgaagcgcaaattaaaagccactcaactatgggcctagttgggtggcttttttgcaaccattacggttttacttctctataagaattatagcatcaatcttcatcattagccaactcaatagaagctcgatatagatcatgttgagctttatccaagctatctaaaagttcgccataaatatcaatatttttcttttcagcaaccaattttttcaaatcttgaagattatcagcaatctgttctgccataactccatagatttcagcttgtttcttcatattgtcaattgtcttaatatcttgtaactccattcctatcactcccatcttacatagttacttaccttagaattccaacgatatatcacagtatctacaacatcatctttttctttttcgtcatccacattaattagatcatcttctttttcgtccagcaataactcatgatgaatctcttttagtaatcccgcaaagcttaattgtcgagttccagccagagcatgctcgagctcgtcaacaacaactaaatcgcctttctcatcatccgtaatataatcataatctttaacttgatatttacttacttcttttgccgaagcaatcaaactattatctttttttgacgcccgaattttcttgatattaacaatcggcttgtagtctaatttccttgctcgcttccataattgtgaccaattctcttgggtaagataatgacccttagaaaaataactagatttcaccattaacaaaatatgtacatgttgatggaaagttagactatctcgattgaccgttatttctgtagatcgcacataaccaagaagatctttttttacatctttatattgaaaaagccgataaattgaacgattcatcgaagtaagattttttctaagctcgcccaactttgagctttcctcagttaatgtcaagaacagaaatatagcactaggatcctttttatatgctgtatctaaaatttgacgcaactcataagaatttttcatcgatcggcgccagctacataatggacaaagtctcgaatgacaaaaccacgtttgatataactttaaaccattatcagttttcgcaaattgaagaacttctccgcactttgaaacgttatgagcttttttaaaatttaaaacctccagatattgggaataagttaagttagcaagttttttctgcttccacggtctaactttcccattactagaggtatcttttagaatttcttttttatacatttcttatacctctaactgcaaaaactacaagcagtcagagttagttatcattgatatattcctaatgacatattataatagggttcgaagttaataacgataactaactcatgcatcgacggccaaatcgaacaactgcatgagttttttatttaattcttacaatgcttattatacaccaaaaagtgttatattttaaaatcctaataaacccagtcatatcaagggtttaacccaatttcaaaaaatggcgttgtttctatatgtatcaagataagaagaaactcgcttacagcgagtttctaaataggggtttcacccctaaaaccctgaccagctgagccagctggaccgcatttcccctcgccgggcgggcgtaaagcggaaaaaatgacttttttctcgctttcgctcaactagggtcttcccgtctcaccatgtcaagcgctgcccttccgctacgctccaggcgtttgtcgcttgacactcacctactaacagcgggcttcgccaccagtctactatctgcaacacggcaacaagattaatcagaatatgcattgaagcagtgcgctccgctccttgtcttgcgacaaagcgagcgcactgcttcaatgcttttttattttctgaattccataactcgataattagaaaaagttagtccgttagaaacgtctgccacgccgaggaagtcattccagtttgaccactcaaatcatcagcgacatcattgagataatcaatcagcaattttctagtttctatctgctttggaatttttaccacagccaaaattgagtcccacttagcaatcacaaaagattttctgacggctttattagccttttccgcactaatatttttcgctttatctcgtttattttgatcggtcgatggtatccgatcgactgttgttacagcatttgacgcaggttgctagataaacgactagaccgaataatttttattccaactatttttgcaaccaaaaaaatcacaaaataatcaaccaataaaacgataactaacttcttgctacatccgattaatttttctccagcctgccagaaatcggcccaaatattgaaatattgaggaaattttgggctgaaaaaaacattaattttttcggcaaacaagccaacacagccaagcagaaaagctaagaaaataagttccagccaattaaaaaatcgccacgaaagagaaaaaaatcggtcaaaataaatcgaacttgatttcattttctagcccccgaagtcttcgataaacggcgccataaaaggaactacacggttttccggttgagtacttgcaagccctaaccccttagaaaagccgtcatgattaacaactgttgtcgactcagctagggccctatatatggatccaattgcaatgatcatcatgacagatctgcgcgcgatcgatatcagcgctttaaatttgcgcatgctagctatagttctagaggtaccggttgttaacgttagccggctacgtatactccggaatattaataggcctaggatgcatatgcttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtg

SEQ ID NO: 13 - DNA, 9013 bp, Artificial Sequence
  pHENOMM
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaatggccatggaggatttattctctccgcagttttgagcttaatcgtctcattaactactgcacttttctccgattggcttcgtcggagaaaataatctaacggcatgaagcgcaaattaaaagccactcaactatgggcctagttgggtggcttttttgcaaccattacggttttacttctctataagaattatagcatcaatcttcatcattagccaactcaatagaagctcgatatagatcatgttgagctttatccaagctatctaaaagttcgccataaatatcaatatttttcttttcagcaaccaattttttcaaatcttgaagattatcagcaatctgttctgccataactccatagatttcagcttgtttcttcatattgtcaattgtcttaatatcttgtaactccattcctatcactcccatcttacatagttacttaccttagaattccaacgatatatcacagtatctacaacatcatctttttctttttcgtcatccacattaattagatcatcttctttttcgtccagcaataactcatgatgaatctcttttagtaatcccgcaaagcttaattgtcgagttccagccagagcatgctcgagctcgtcaacaacaactaaatcgcctttctcatcatccgtaatataatcataatctttaacttgatatttacttacttcttttgccgaagcaatcaaactattatctttttttgacgcccgaattttcttgatattaacaatcggcttgtagtctaatttccttgctcgcttccataattgtgaccaattctcttgggtaagataatgacccttagaaaaataactagatttcaccattaacaaaatatgtacatgttgatggaaagttagactatctcgattgaccgttatttctgtagatcgcacataaccaagaagatctttttttacatctttatattgaaaaagccgataaattgaacgattcatcgaagtaagattttttctaagctcgcccaactttgagctttcctcagttaatgtcaagaacagaaatatagcactaggatcctttttatatgctgtatctaaaatttgacgcaactcataagaatttttcatcgatcggcgccagctacataatggacaaagtctcgaatgacaaaaccacgtttgatataactttaaaccattatcagttttcgcaaattgaagaacttctccgcactttgaaacgttatgagcttttttaaaatttaaaacctccagatattgggaataagttaagttagcaagttttttctgcttccacggtctaactttcccattactagaggtatcttttagaatttcttttttatacatttcttatacctctaactgcaaaaactacaagcagtcagagttagttatcattgatatattcctaatgacatattataatagggttcgaagttaataacgataactaactcatgcatcgacggccaaatcgaacaactgcatgagttttttatttaattcttacaatgcttattatacaccaaaaagtgttatattttaaaatcctaataaacccagtcatatcaagggtttaacccaatttcaaaaaatggcgttgtttctatatgtatcaagataagaagaaactcgcttacagcgagtttctaaataggggtttcacccctaaaaccctgaccagctgagccagctggaccgcatttcccctcgccgggcgggcgtaaagcggaaaaaatgacttttttctcgctttcgctcaactagggtcttcccgtctcaccatgtcaagcgctgcccttccgctacgctccaggcgtttgtcgcttgacactcacctactaacagcgggcttcgccaccagtctactatctgcaacacggcaacaagattaatcagaatatgcattgaagcagtgcgctccgctccttgtcttgcgacaaagcgagcgcactgcttcaatgcttttttattttctgaattccataactcgataattagaaaaagttagtccgttagaaacgtctgccacgccgaggaagtcattccagtttgaccactcaaatcatcagcgacatcattgagataatcaatcagcaattttctagtttctatctgctttggaatttttaccacagccaaaattgagtcccacttagcaatcacaaaagattttctgacggctttattagccttttccgcactaatatttttcgctttatctcgtttattttgatcggtcgatggtatccgatcgactgttgttacagcatttgacgcaggttgctagataaacgactagaccgaataatttttattccaactatttttgcaaccaaaaaaatcacaaaataatcaaccaataaaacgataactaacttcttgctacatccgattaatttttctccagcctgccagaaatcggcccaaatattgaaatattgaggaaattttgggctgaaaaaaacattaattttttcggcaaacaagccaacacagccaagcagaaaagctaagaaaataagttccagccaattaaaaaatcgccacgaaagagaaaaaaatcggtcaaaataaatcgaacttgatttcattttctagcccccgaagtcttcgataaacggcgccataaaaggaactacacggttttccggttgagtacttgcaagccctaaccccttagaaaagccgtcatgattaacaactgttgtcgacttttttttgataagttaagggatgcagtttatgcaccccttaacttatcattattagtgtaaacaaggaaggatatcttgtacggcttgaacaattacaccaccagcagcgatatcagcagaaatacgagcaatatgttcgtcaagtccttgttcgttatcgttccaaatatatggccgatctgatgtaggtttttggccgacaacatgacgtactgctgaataaagacgttctgtagctggacttaaacaagcacgagcatcataatggcccgtctttttgtaagtccgtaaatcaacagcttgtactccaaacattaatgcaatagctacgtagttttgaaaaatatcaacagaccgtcgtgctaacgtagcagaagtgtatccttgagagttaatattttggttaaattgttcggcgtgtgtaggaaagcgatcagcaattgaatttccgtaaaaagtcaacaatggcataattgagttgccacaaatttgcaaacccttaagacccatgtttacctttcgttcccggtttcccaataatgatggtggaagtccgttactgaattctggactggccaaaagtgcaatttggacatccaaatgctttgcaagaagaccaatgtagtaacgtaagtggtccatgcccatgccaacatattgacccaaaaagttaccgccatggtatgaggcttggttatcaacgtcaattaatggattgtcagtgacactattaatttcgatttcaatttgcttagcaatttgtgaaattccatcaacaataggacccaaatattgtggtaagcaacgtaaactatagcggtcttgaataagttcatgatcacgataatcatgcttaccatctaattcgtcgcgaacaagttgactattagctaaaagagaaatcatttgatcagcagcccaaagttgacctgggtgaggtttagaattgtgaatgaatgggtggaaactttgatttgtgccattaagtgcttgaatatctaaggcatgaacacccatggcaatagcagtaagaatttgagtatcataaacgcaattagcagcaattcccgtcataactgatgtaccattcatcatagcaagaccttcttttggaagtaatgtaagtggtgataaatttaattgtcgaagagctgttggggcatccatttctttaccattaaaatcaactttgaatgatggatccaaaccaattaaagaacctgtaatataagataaaggaacaagatctccacttgcaccaattgatccgaattcgtaaacataaggtgtaacaccggcgttaaggaaaatttccatacgcttaattaattcaagacgaataccagaagcgccacgcatgtgtgaatttgcacgtaataacatagcagcacgaacatctgctaatggaagtttattaccagctcctgttttaagaaaccaaacaagattagtttgaagttcagaagcttgttcacgactaatagcgacgtttgccattccaccgaatcctgaagtaacaccataaattggttctcctgattcaacagcgttattgatatagtcgcaactagcttgaatgccttgtaaaatgtcagtgttgttagttaaactaactaaagtaccattgcgtgcaacccgtgcaacatcgttaatagtaagcttttgattaccaatgataacattagcacttgagtttccagtaaatgagaattgttgagaactagtcttagattgtgcttgagataaagtcttcatttatacctactccttcttaattacaaatttttagcatctaatttaacttcaattcctattatacaaaattttaagatactgcactatcaacacactcttgaacatatgcatcctaggtcgtctattaatacttccaggagtatacgtagccaggctaacgttaatcaaccaggtatcctctagaactatagctagcatgcctatatatggatccaattgcaatgatcatcatgacagatctgcgcgcgatcgatatcagcgctttaaatttgcgcatgctagctatagttctagaggtaccggttgttaacgttagccggctacgtatactccggaatattaataggcctaggatgcatatgcttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagataaattattaggtatactactgacagcttccaagcatatggcggccgcctgcagctggcgccatcgatacgcgtacgtcgcgaccgcggacatgtacagagctcgagaagtactagtggccacgtgggccgtgcaccttaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 14 - DNA, 9142 bp, Artificial Sequence
  pHENOMM-sec
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgaatggccatggaggatttattctctccgcagttttgagcttaatcgtctcattaactactgcacttttctccgattggcttcgtcggagaaaataatctaacggcatgaagcgcaaattaaaagccactcaactatgggcctagttgggtggcttttttgcaaccattacggttttacttctctataagaattatagcatcaatcttcatcattagccaactcaatagaagctcgatatagatcatgttgagctttatccaagctatctaaaagttcgccataaatatcaatatttttcttttcagcaaccaattttttcaaatcttgaagattatcagcaatctgttctgccataactccatagatttcagcttgtttcttcatattgtcaattgtcttaatatcttgtaactccattcctatcactcccatcttacatagttacttaccttagaattccaacgatatatcacagtatctacaacatcatctttttctttttcgtcatccacattaattagatcatcttctttttcgtccagcaataactcatgatgaatctcttttagtaatcccgcaaagcttaattgtcgagttccagccagagcatgctcgagctcgtcaacaacaactaaatcgcctttctcatcatccgtaatataatcataatctttaacttgatatttacttacttcttttgccgaagcaatcaaactattatctttttttgacgcccgaattttcttgatattaacaatcggcttgtagtctaatttccttgctcgcttccataattgtgaccaattctcttgggtaagataatgacccttagaaaaataactagatttcaccattaacaaaatatgtacatgttgatggaaagttagactatctcgattgaccgttatttctgtagatcgcacataaccaagaagatctttttttacatctttatattgaaaaagccgataaattgaacgattcatcgaagtaagattttttctaagctcgcccaactttgagctttcctcagttaatgtcaagaacagaaatatagcactaggatcctttttatatgctgtatctaaaatttgacgcaactcataagaatttttcatcgatcggcgccagctacataatggacaaagtctcgaatgacaaaaccacgtttgatataactttaaaccattatcagttttcgcaaattgaagaacttctccgcactttgaaacgttatgagcttttttaaaatttaaaacctccagatattgggaataagttaagttagcaagttttttctgcttccacggtctaactttcccattactagaggtatcttttagaatttcttttttatacatttcttatacctctaactgcaaaaactacaagcagtcagagttagttatcattgatatattcctaatgacatattataatagggttcgaagttaataacgataactaactcatgcatcgacggccaaatcgaacaactgcatgagttttttatttaattcttacaatgcttattatacaccaaaaagtgttatattttaaaatcctaataaacccagtcatatcaagggtttaacccaatttcaaaaaatggcgttgtttctatatgtatcaagataagaagaaactcgcttacagcgagtttctaaataggggtttcacccctaaaaccctgaccagctgagccagctggaccgcatttcccctcgccgggcgggcgtaaagcggaaaaaatgacttttttctcgctttcgctcaactagggtcttcccgtctcaccatgtcaagcgctgcccttccgctacgctccaggcgtttgtcgcttgacactcacctactaacagcgggcttcgccaccagtctactatctgcaacacggcaacaagattaatcagaatatgcattgaagcagtgcgctccgctccttgtcttgcgacaaagcgagcgcactgcttcaatgcttttttattttctgaattccataactcgataattagaaaaagttagtccgttagaaacgtctgccacgccgaggaagtcattccagtttgaccactcaaatcatcagcgacatcattgagataatcaatcagcaattttctagtttctatctgctttggaatttttaccacagccaaaattgagtcccacttagcaatcacaaaagattttctgacggctttattagccttttccgcactaatatttttcgctttatctcgtttattttgatcggtcgatggtatccgatcgactgttgttacagcatttgacgcaggttgctagataaacgactagaccgaataatttttattccaactatttttgcaaccaaaaaaatcacaaaataatcaaccaataaaacgataactaacttcttgctacatccgattaatttttctccagcctgccagaaatcggcccaaatattgaaatattgaggaaattttgggctgaaaaaaacattaattttttcggcaaacaagccaacacagccaagcagaaaagctaagaaaataagttccagccaattaaaaaatcgccacgaaagagaaaaaaatcggtcaaaataaatcgaacttgatttcattttctagcccccgaagtcttcgataaacggcgccataaaaggaactacacggttttccggttgagtacttgcaagccctaaccccttagaaaagccgtcatgattaacaactgttgtcgacttttttttgataagttaagggatgcagtttatgcaccccttaacttatcattattagtgtaaacaaggaaggatatcttgtacggcttgaacaattacaccaccagcagcgatatcagcagaaatacgagcaatatgttcgtcaagtccttgttcgttatcgttccaaatatatggccgatctgatgtaggtttttggccgacaacatgacgtactgctgaataaagacgttctgtagctggacttaaacaagcacgagcatcataatggcccgtctttttgtaagtccgtaaatcaacagcttgtactccaaacattaatgcaatagctacgtagttttgaaaaatatcaacagaccgtcgtgctaacgtagcagaagtgtatccttgagagttaatattttggttaaattgttcggcgtgtgtaggaaagcgatcagcaattgaatttccgtaaaaagtcaacaatggcataattgagttgccacaaatttgcaaacccttaagacccatgtttacctttcgttcccggtttcccaataatgatggtggaagtccgttactgaattctggactggccaaaagtgcaatttggacatccaaatgctttgcaagaagaccaatgtagtaacgtaagtggtccatgcccatgccaacatattgacccaaaaagttaccgccatggtatgaggcttggttatcaacgtcaattaatggattgtcagtgacactattaatttcgatttcaatttgcttagcaatttgtgaaattccatcaacaataggacccaaatattgtggtaagcaacgtaaactatagcggtcttgaataagttcatgatcacgataatcatgcttaccatctaattcgtcgcgaacaagttgactattagctaaaagagaaatcatttgatcagcagcccaaagttgacctgggtgaggtttagaattgtgaatgaatgggtggaaactttgatttgtgccattaagtgcttgaatatctaaggcatgaacacccatggcaatagcagtaagaatttgagtatcataaacgcaattagcagcaattcccgtcataactgatgtaccattcatcatagcaagaccttcttttggaagtaatgtaagtggtgataaatttaattgtcgaagagctgttggggcatccatttctttaccattaaaatcaactttgaatgatggatccaaaccaattaaagaacctgtaatataagataaaggaacaagatctccacttgcaccaattgatccgaattcgtaaacataaggtgtaacaccggcgttaaggaaaatttccatacgcttaattaattcaagacgaataccagaagcgccacgcatgtgtgaatttgcacgtaataacatagcagcacgaacatctgctaatggaagtttattaccagctcctgttttaagaaaccaaacaagattagtttgaagttcagaagcttgttcacgactaatagcgacgtttgccattccaccgaatcctgaagtaacaccataaattggttctcctgattcaacagcgttattgatatagtcgcaactagcttgaatgccttgtaaaatgtcagtgttgttagttaaactaactaaagtaccattgcgtgcaacccgtgcaacatcgttaatagtaagcttttgattaccaatgataacattagcacttgagtttccagtaaatgagaattgttgagaactagtcttagattgtgcttgagataaagtcttcatttatacctactcctatcaactgaagaagcatcaacaaccataatattttggcttacgccccaaaaactaataacgaccaataatggtaatagccacacaggaagtccgttacggtgatgtttacctgtattttcagtatacattcttaattacaaatttttagcatctaatttaacttcaattcctattatacaaaattttaagatactgcactatcaacacactcttgaacatatgcatcctaggtcgtctattaatacttccaggagtatacgtagccaggctaacgttaatcaaccaggtatcctctagaactatagctagcatgcctatatatggatccaattgcaatgatcatcatgacagatctgcgcgcgatcgatatcagcgctttaaatttgcgcatgctagctatagttctagaggtaccggttgttaacgttagccggctacgtatactccggaatattaataggcctaggatgcatatgcttagaagcaaacttaagagtgtgttgatagtgcagtatcttaaaattttgtataataggaattgaagttaaattagatgctaaaaatttgtaattaagaaggagtgattacatgaacaaaaatataaaatattctcaaaactttttaacgagtgaaaaagtactcaaccaaataataaaacaattgaatttaaaagaaaccgataccgtttacgaaattggaacaggtaaagggcatttaacgacgaaactggctaaaataagtaaacaggtaacgtctattgaattagacagtcatctattcaacttatcgtcagaaaaattaaaactgaatactcgtgtcactttaattcaccaagatattctacagtttcaattccctaacaaacagaggtataaaattgttgggagtattccttaccatttaagcacacaaattattaaaaaagtggtttttgaaagccatgcgtctgacatctatctgattgttgaagaaggattctacaagcgtaccttggatattcaccgaacactagggttgctcttgcacactcaagtctcgattcagcaattgcttaagctgccagcggaatgctttcatcctaaaccaaaagtaaacagtgtcttaataaaacttacccgccataccacagatgttccagataaatattggaagctatatacgtactttgtttcaaaatgggtcaatcgagaatatcgtcaactgtttactaaaaatcagtttcatcaagcaatgaaacacgccaaagtaaacaatttaagtaccgttacttatgagcaagtattgtctatttttaatagttatctattatttaacgggaggaaataattctatgagtcgcttttgtaaatttggaaagttacacgttactaaagggaatgtagataaattattaggtatactactgacagcttccaagcatatggcggccgcctgcagctggcgccatcgatacgcgtacgtcgcgaccgcggacatgtacagagctcgagaagtactagtggccacgtgggccgtgcaccttaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 15 - DNA, 1963 bp, Artificial Sequence
  synthesized
gcatgctagctatagttctagaggatacctggttgattaacgttagcctggctacgtatactcctggaagtattaatagacgacctaggatgcatatgttcaagagtgtgttgatagtgcagtatcttaaaattttgtataataggaattgaagttaaattagatgctaaaaatttgtaattaagaaggagtaggtataaatgaagactttatctcaagcacaatctaagactagttctcaacaattctcatttactggaaactcaagtgctaatgttatcattggtaatcaaaagcttactattaacgatgttgcacgggttgcacgcaatggtactttagttagtttaactaacaacactgacattttacaaggcattcaagctagttgcgactatatcaataacgctgttgaatcaggagaaccaatttatggtgttacttcaggattcggtggaatggcaaacgtcgctattagtcgtgaacaagcttctgaacttcaaactaatcttgtttggtttcttaaaacaggagctggtaataaacttccattagcagatgttcgtgctgctatgttattacgtgcaaattcacacatgcgtggcgcttctggtattcgtcttgaattaattaagcgtatggaaattttccttaacgccggtgttacaccttatgtttacgaattcggatcaattggtgcaagtggagatcttgttcctttatcttatattacaggttctttaattggtttggatccatcattcaaagttgattttaatggtaaagaaatggatgccccaacagctcttcgacaattaaatttatcaccacttacattacttccaaaagaaggtcttgctatgatgaatggtacatcagttatgacgggaattgctgctaattgcgtttatgatactcaaattcttactgctattgccatgggtgttcatgccttagatattcaagcacttaatggcacaaatcaaagtttccacccattcattcacaattctaaacctcacccaggtcaactttgggctgctgatcaaatgatttctcttttagctaatagtcaacttgttcgcgacgaattagatggtaagcatgattatcgtgatcatgaacttattcaagaccgctatagtttacgttgcttaccacaatatttgggtcctattgttgatggaatttcacaaattgctaagcaaattgaaatcgaaattaatagtgtcactgacaatccattaattgacgttgataaccaagcctcataccatggcggtaactttttgggtcaatatgttggcatgggcatggaccacttacgttactacattggtcttcttgcaaagcatttggatgtccaaattgcacttttggccagtccagaattcagtaacggacttccaccatcattattgggaaaccgggaacgaaaggtaaacatgggtcttaagggtttgcaaatttgtggcaactcaattatgccattgttgactttttacggaaattcaattgctgatcgctttcctacacacgccgaacaatttaaccaaaatattaactctcaaggatacacttctgctacgttagcacgacggtctgttgatatttttcaaaactacgtagctattgcattaatgtttggagtacaagctgttgatttacggacttacaaaaagacgggccattatgatgctcgtgcttgtttaagtccagctacagaacgtctttattcagcagtacgtcatgttgtcggccaaaaacctacatcagatcggccatatatttggaacgataacgaacaaggacttgacgaacatattgctcgtatttctgctgatatcgctgctggtggtgtaattgttcaagccgtacaagatatccttccttgtttacactaataatgataagttaaggggtgcataaactgcatcccttaacttatcaaaaaaaagtcgac